(19) United States
(12) Patent Application Publication
(10) Pub. No.: US 2009/0177239 A1
(43) Pub. Date: Jul. 9, 2009

(54) CERVICAL PLATE INSTRUMENT KIT

(76) Inventor: Michael Castro, Uxbridge, MA (US)

Correspondence Address: CARR LLP (IST), 670 FOUNDERS SQUARE, 900 JACKSON STREET, DALLAS, TX, 75202, US

(21) Appl. No.: 12/187,252

(22) Filed: Aug. 6, 2008

Related U.S. Application Data
(60) Provisional application No. 60/954,226, filed on Aug. 6, 2007.

(51) Int. Cl. A61B 17/56

(52) U.S. Cl. 606/86 B; 606/86 R

(57) ABSTRACT

The present invention provides an apparatus, method and kit for placing and securing an implant to vertebral bodies of a spine. The apparatus may have an implant engaging base having implant engaging limbs coupled to a handle and an aligning member pivotally coupled to the handle by a spring loaded collar where the aligning member may be rotated and locked into fixed positions. The aligning member may receive a cannula for guiding instruments and tools into the surgical field. A method is presented which includes turning locking screws to frictionally engage rounded screws to secure the implant to the vertebral bodies. Flats on the head of the locking screws may be turned by a screwdriver having corresponding flats dispositioned to align in a coaxial manner to the flats on the heads of the locking screws. A kit is presented including the apparatus, the screws, and the screwdriver.

### CROSS-REFERENCE TO RELATED APPLICATIONS

This application relates to, and claims the benefit of the filing date of, co-pending U.S. provisional patent application Ser. No. 60/954,226 entitled CERVICAL PLATE INSTRUMENT KIT, filed Aug. 6, 2007, the entire contents of which are incorporated herein by reference for all purposes.

### FIELD OF THE INVENTION

This disclosure relates to devices, instruments, apparatuses, and methods for performing subcutaneous and percutaneous surgery, more particularly, to devices, instruments, apparatuses, and methods for performing minimally invasive spinal surgery.

### BACKGROUND AND SUMMARY

Patients suffering from orthopedic injuries, deformities, or degenerative diseases often need surgery to stabilize an internal structure, promote healing, or relieve pain. Surgeries to correct spinal problems often involve placing implants, such as braces, rods, and various implants between one or more of the patient's vertebrae, anchored into the vertebrae pedicles by fasteners such as screws or hooks. Proper orientation and alignment of fasteners and implants, such as stabilization plates are critical in surgical procedures to correct damaged areas of the spine, especially in the cervical spine, because there is only a small amount of bone available for receiving fasteners to anchor any implants. Whereas traditional stabilizing plates require bone anchors, such as anchoring screws, to penetrate both the anterior and posterior bone cortices in order to provide a sufficient anchor for a stabilizing plate, plates anchored into vertebral bodies in the cervical spine are preferably only anchored into only the anterior bone cortex so as not to risk penetration of the spinal cord. In addition to precision and accuracy, less invasion into the muscle and tissue surrounding the surgical field possible is best to avoid the risk of damage and further injury to the surrounding tissue and minimize post-surgery recovery.

Traditional procedures for placing implants into the cervical spine require placing the implant, such as a cervical plate and then securing the implant to the affected vertebral bodies in order to maintain the implant in a substantially permanent position. An implant generally has multiple pairs of orifices therein for receiving multiple fasteners to secure the plate to one or more adjacent vertebral bodies. A tool such as a cannula or drill guide is generally inserted over one orifice to drill a hole into the bone cortex of a vertebral body for receiving a fastener such as a screw. A tap is then inserted to tap the hole and the fastener is then inserted and secured into the vertebral body thereby partially securing the cervical plate. The cannula or drill guide must then be removed and repositioned over the other orifice to place an adjacent fastener. The cannula or drill guide must be inserted, removed, and replaced for each orifice on the cervical implant displacing and potentially damaging surrounding tissue each time.

The present invention comprises a kit comprising tools, instruments, and an implant for correcting spinal problems in the cervical spine which overcomes the foregoing and other difficulties which have long since characterized the prior art. Certain aspects of the present invention provide methods and apparatuses used in percutaneous and subcutaneous surgical techniques for correcting spinal defects and injuries with minimal invasion and disruption to the tissue surrounding the affected vertebrae. There is disclosed a kit comprising a stabilizing plate, fastening and drilling tools, and a placement and guidance apparatus for placing and securing the stabilizing plate into at least two vertebrae of the cervical spine comprising an implant engaging base with a handle and an instrument guide or cannula pivotally attached thereto.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is a perspective view of one embodiment of a placement and guidance apparatus used in the placement and securing of a stabilizing plate incorporating various aspects of the present invention;

FIG. 1A is a section view of the placement and guidance apparatus shown in FIG. 1;

FIG. 2 is a top view of an implant which may be placed and secured utilizing the placement and guidance apparatus shown in FIG. 1;

FIG. 3 is a perspective view showing the placement and guidance apparatus shown in FIG. 1 coupled with the implant shown in FIG. 2;

FIG. 3A is a top view of the placement and guidance apparatus shown in FIG. 1 coupled with the implant shown in FIG. 2 illustrating various aspects of the present invention;

FIG. 4 illustrates various tools and instruments incorporating various aspects of the present invention which may be combined into a kit;

FIG. 4A-4B are plan views of fasteners that may be used in an embodiment of the invention;

FIG. 5 is a flowchart illustrating one embodiment of a method for using the tools and instruments of the kit of FIG. 4; and

FIG. 6 is a perspective view of an alternate embodiment of a placement and guidance apparatus.

### DETAILED DESCRIPTION

In the following discussion, numerous specific details are set forth to provide a thorough understanding of the present invention. However, those skilled in the art will appreciate that the present invention may be practiced without such specific details.

The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

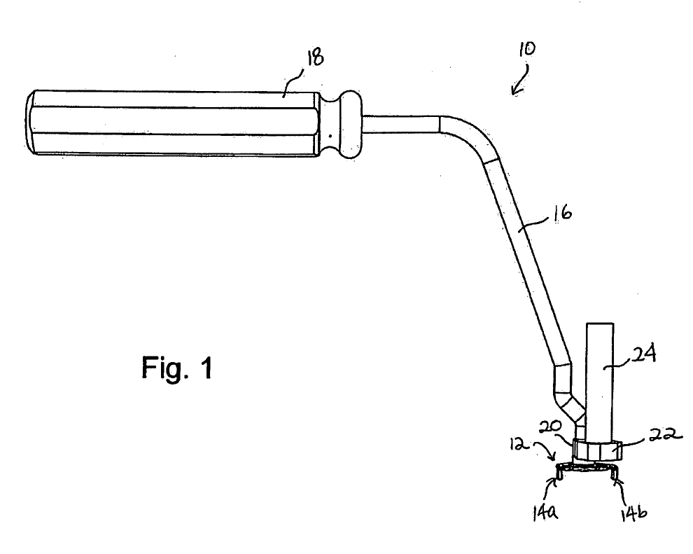

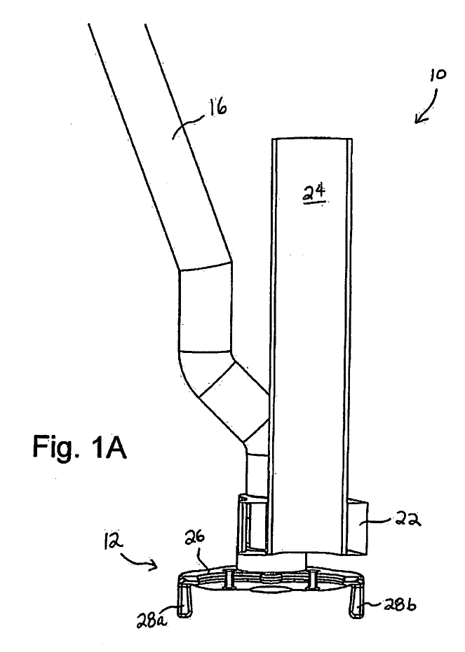

Referring to FIG. 1 there is shown one embodiment of a placement and guidance apparatus 10 for placing and securing an implant such as a cervical stabilizing plate into at least two vertebral bodies of the cervical spine. In some embodiments, the placement and guidance apparatus 10 may comprise an implant engaging base 12 having implant engaging members 14a and 14b on opposing sides thereof for engaging the implant during placement into a surgical field and thereafter stabilizing the placement and guidance apparatus 10 against the implant while the vertebral bodies are prepared for and receive fasteners for fastening the implant thereto. The apparatus may further comprise a handle 16 extending above the implant engaging base 12. The handle 16 may be rotatably coupled with the implant engaging base 12 such that the handle 16 may rotate at least 180 degrees for enabling manipulation of the placement and guidance apparatus 10 by a user on either side of the patient and thus the surgical field. The handle 16 may further comprise a grip 18 thereon for ergonomic comfort and ease of manipulation for a user of the placement and guidance apparatus 10. Pivotally coupled about the handle 16 above the implant engaging base 12 may be a pivoting member such as a spring-loaded collar 20 having aligning member 22 extending therefrom. In some embodiments the aligning member 22 may comprise a cannula 24 for guiding tools and instruments such as awls, drills, taps, screwdrivers, and fasteners therethrough. The spring-loaded collar 20 may be pivoted about the handle enabling axial alignment of the aligning member 22 and cannula 24 over orifices in the implant, the orifices for receiving fasteners for securing the implant to vertebral bodies. Once aligned above each orifice, the cannula 24 may thereafter be used to guide instruments into the surgical field for fastening the implant to vertebral bodies with less intrusion into surrounding tissue and therefore less damage to tissue surrounding the surgical field than previously experienced in traditional spine correction surgeries. In some embodiments, the spring-loaded collar 20 may be spring-loaded to facilitate locking the aligning member 22 into position over a selected orifice while instruments are inserted through the cannula 24 and thereafter releasing the lock on the position of the aligning member 22 such that the aligning member 22 may be pivoted over to an adjacent orifice.

Referring now to FIG. 1A, there is shown a section view of the placement and guidance apparatus 10 further illustrating various aspects of function and construction of the present invention. The implant engaging base 12 of the placement and guidance apparatus 10 may comprise a saddle 26 which straddles an implant. In some embodiments the saddle 26 may comprise implant engaging limbs 28a and 28b on opposing sides thereof for engaging the implant during placement into a surgical field and thereafter stabilizing the apparatus 10 against the implant while the vertebral bodies to be stabilized by the implant are prepared for and receive fasteners for fastening the implant to the prepared vertebral bodies.

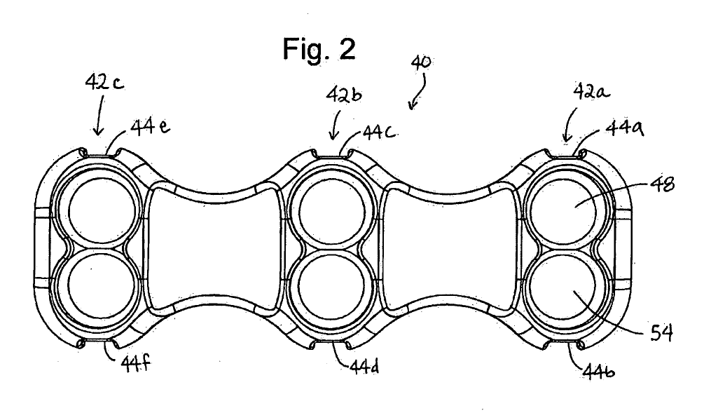

Referring now to FIG. 2, there is shown a cervical stabilizing plate 40 which may be implanted and thereafter secured to two or more adjacent vertebral bodies using the placement and guidance apparatus 10 shown in FIG. 1. The cervical stabilizing plate 40 may comprise multiple pairs of orifices 42a-42c for receiving fasteners therethrough to fasten the cervical stabilizing plate 40 to the vertebral bodies. Adjacent to each pair of orifices 42a-42c on opposing sides thereof are indentions 44a-44f which may enable engagement by the limbs 28a and 28b of the saddle 26 (FIG. 1A) such that the placement and guidance apparatus 10 (FIG. 1A) may be used to insert and position the cervical stabilizing plate 40 over the vertebral bodies in the surgical field and to maintain the position of the cervical stabilizing plate 40 while guiding instruments through the cannula 24 for preparing the vertebral body to receive a fastener and for thereafter inserting and securing the fastener to the vertebral body.

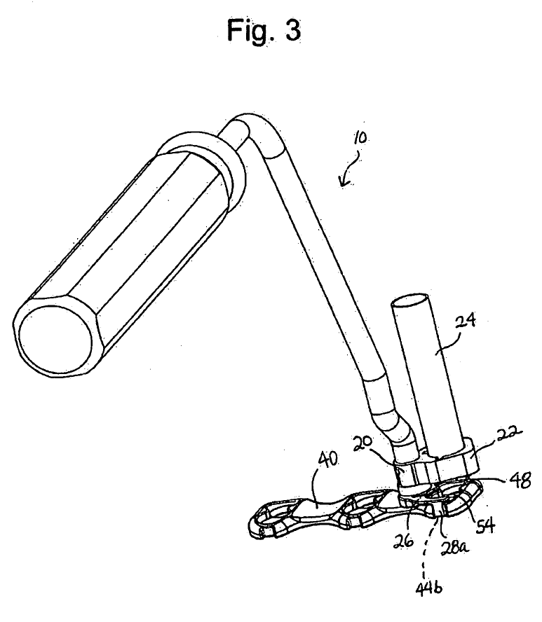

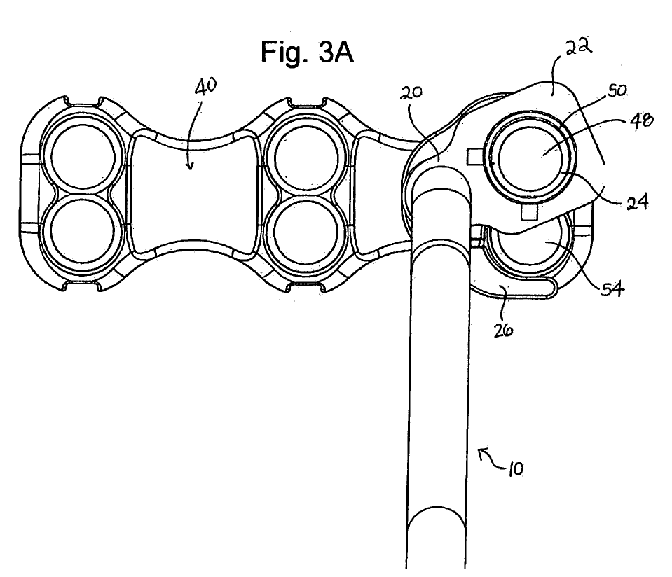

Referring now to FIGS. 3 and 3A there are shown different views of the placement and guidance apparatus 10 (FIG. 1) coupled with the cervical stabilizing plate 40 illustrating certain aspects of the invention. Referring specifically to FIG. 3, there is shown a perspective view of the placement and guidance apparatus 10 and the cervical stabilizing plate 40. The placement and guidance apparatus 10 is illustrated with the cervical stabilizing plate 40 and cannula 24 positioned over a first orifice 48 of the pair of orifices 42a (FIG. 2). The spring-loaded collar 20 may enable locking the aligning member 22 and cannula 24 into a first substantially fixed position relative to the cervical stabilizing plate 40 comprising the pair of orifices 42 such that the cannula 24 is axially aligned with the first orifice 48 during insertion of tools and instruments into the surgical field for preparing the vertebral body for receiving a fastener and thereafter inserting and securing the fastener. The aligning member 22 and cannula 24 may then be released from the first position and be repositioned and locked into a second substantially fixed position relative to the cervical stabilizing plate 40 such that the cannula 24 may be axially aligned with a second orifice 54 during insertion of tools and instruments into the surgical field for preparing the vertebral body for and receiving a fastener.

In certain embodiments, the saddle 26 may grasp the cervical stabilizing plate 40 by the limbs 28a and 28b (FIG. 1A) engaging opposing indentions 44a and 44b (FIG. 2). In this manner, the placement and guidance apparatus 10 may be used to insert and position the cervical stabilizing plate 40 into the surgical field and maintain the placement and guidance apparatus 10 and cannula 24 in a substantially fixed position with respect to the cervical stabilizing plate 40 during further manipulation of instruments and apparatus into the surgical field.

Referring now to FIG. 3A, there is shown a top view of the placement and guidance apparatus 10 and the cervical stabilizing plate 40 illustrating another aspect of the invention. The placement and guidance apparatus 10 is shown with the spring-loaded collar 20 rotated, positioning the aligning member 22 and cannula 24 over the first orifice 48 of the pair of orifices 42a (FIG. 2) such that the cannula 24 is axially aligned with the orifice 48 therebelow. FIG. 3A also illustrates the construction of the cannula 24 with respect to the cervical stabilizing plate 40 which may be incorporated into one embodiment of the invention. The cervical stabilizing plate 40 may comprise a locking ring 50 welded therein for receiving and securing the cannula 24 into the cervical stabilizing plate 40.

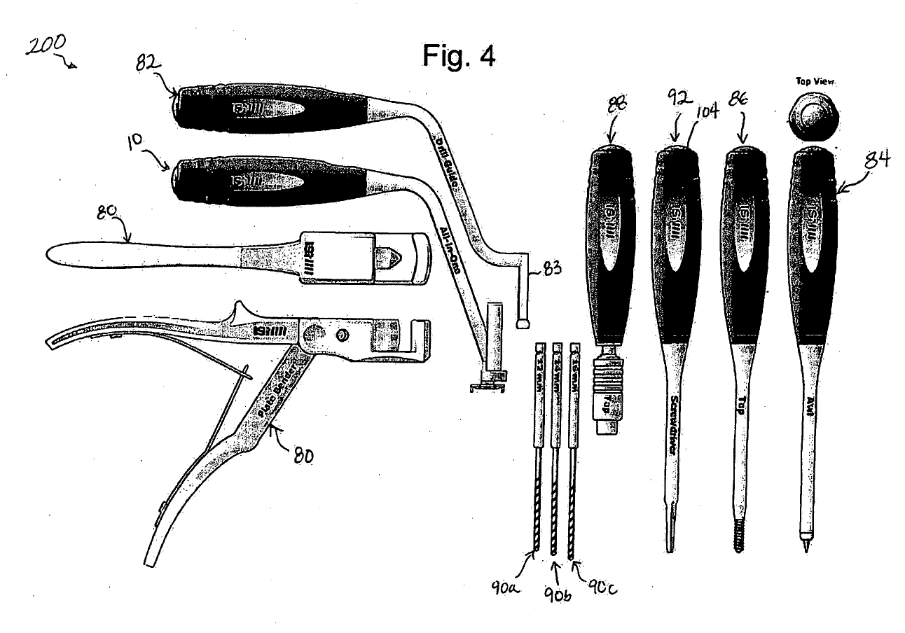

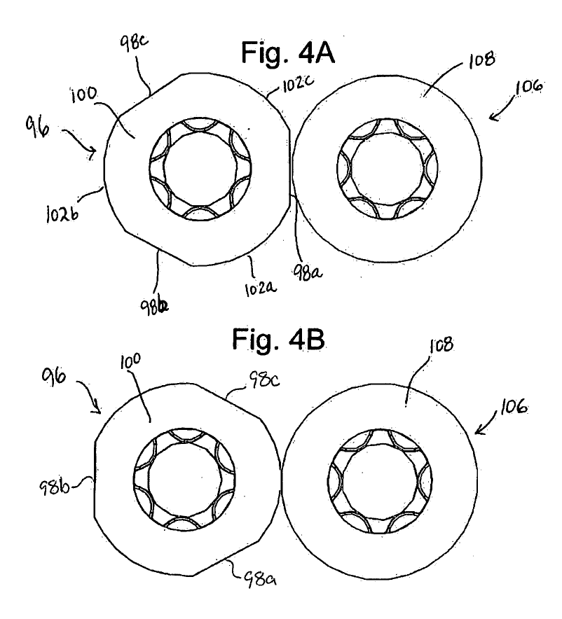

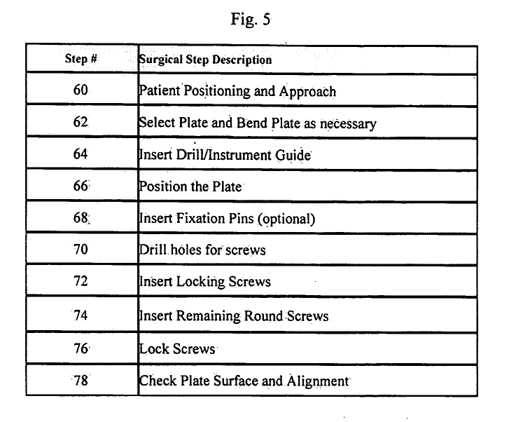

Referring now to FIGS. 4 and 4A, there are shown instruments and tools which may comprise the kit of the present invention. Referring to FIG. 5, there is shown a flowchart of a surgical procedure for inserting a cervical stabilizing plate 40 which may be performed utilizing the kit according to the present invention. Accordingly, the tools and instruments shown in FIGS. 4 and 4A and the placement and guidance apparatus 10 and cervical stabilizing plate 40 shown in FIGS. 1 through 3A may be utilized during a surgical procedure to place one or more implants into the cervical spine for correcting damage or injury thereto as described in conjunction with FIG. 5. Step 60) “Patient Positioning and Approach” may comprise positioning the patent according to a surgeon's preference and preparing the surgical field, a step well known to those skilled in the fields of orthopedic and/or neurosurgery. Step 62) “Select Plate and Bend and Necessary” may comprise selecting an appropriate size stabilizing plate such as cervical stabilizing plate 40 according to the patient's anatomy and number of cervical vertebrae needing correction. The stabilizing plate may be bent prior to positioning in the patient using a plate bender such as plate bender 80 shown in FIG. 4, shown both from a side and top view.

Step 64) “Insert Drill Guide” may comprise positioning a drill guide such as drill guide 82 shown in FIG. 4 within the surgical field for assisting with guidance of the plate and other instruments and tools into the surgical field. A surgical field for a cervical vertebral procedure is traditionally very small due to the size of cervical vertebrae and the delicate surrounding nerves, tissue, and muscle. Accordingly, the drill guide 82 may comprise a cannula 83 thereon and may thereafter be used in later steps for guiding an awl such as awl 84, a tap such as taps 86 and 88, drill bits such as drill bits 90a-90c, and a screwdriver such as screwdriver 92 shown in FIG. 4. The cannula 83 of drill guide 82 may comprise a fixed angle cannula or a variable angle cannula. Step 66) “Position the Plate” may comprise inserting a plate such as cervical stabilizing plate 40 into the surgical field utilizing a plate holder and positioning the plate above the vertebral bodies to which the cervical stabilizing plate 40 will be secured. Steps 64 and 66 may both be performed using the placement and guidance apparatus 10 wherein the placement and guidance apparatus 10 may be used to position the cervical stabilizing plate 40 into the surgical field and for guiding instruments such as the awl 84, taps 86 and 88, drill bits 90a-90c, and screwdriver 92 for preparing the vertebral body for fasteners such as screws into the surgical field without disrupting any tissue surrounding tissue surrounding the surgical field.

Step 68) “Insert Fixation Pins” may comprise temporarily coupling a cervical stabilizing plate 40 to the vertebral bodies with temporary holding pins while the vertebral bodies are prepared for fasteners which are then inserted through the plate thereby securing the plate to the vertebral bodies. Step 68 may not be necessary when utilizing the placement and guidance apparatus 10 in conjunction with a stabilizing plate similar to cervical stabilizing plate 40 as the placement and guidance apparatus 10 may be utilized to position the stabilizing plate and thereafter maintain the stabilizing plate into a substantially fixed position until the stabilizing plate is secured to at least one vertebral body. Step 70) “Drill holes for screws” may comprise preparing the vertebral bodies for fasteners such as screws. This step may comprise first preparing an initial hole using awl 84 having a point on one end thereof. The prepared hole may then be drilled to a predetermined depth and diameter using one of the drill bits 90a-90c. The drilled hole may then be tapped using either tap 86 or 88 according to the diameter of the fastener to be inserted therein. Step 72) “Insert locking screws” may comprise inserting a locking screw 96 having 3 flats 98a-98c on screw head 100 as shown in FIG. 4A with rounds 102a-102c therebetween. The screw 96 may be inserted into one orifice of the pair of orifices in the cervical stabilizing plate 40 and turned using screwdriver 92 (FIG. 4) such that the one of the flats is faced inwardly toward the adjacent orifice. The screwdriver 92 likewise may have similar flats on its handle head 104 such that a surgeon may adjust the turn of the locking screw without requiring visual access of the screw head 100. The direction of the flats 98a-98c of the locking screw 96 may be determined by aligning the flats of the handle of the screwdriver 92 with the flats 98a-98c of the locking screw 96. When the flat is positioned inwardly, the locking screw 96 may be in a non-interference position ready to accept a round screw adjacent thereto. The drill guide 82 may be thereafter removed and repositioned over the adjacent orifice, or alternatively, the aligning member 22 and cannula 24 of the placement and guidance apparatus 10 may be pivoted over the adjacent orifice and step 70 may be repeated to further prepare the vertebral body for another fastener. Step 74) “Insert round screws” may comprise inserting round screws 106 having round heads 108 as shown in FIG. 4A adjacent to the locking screw 96. Step 76) “Lock screws” may comprise again accessing the locking screw 96 and turning the head 100 into a locked position defined by one of the rounds 102a-102c between the flats 98a-98c such that the head 100 of the locking screw 96 may frictionally engage the round head 108 of the round screw 106 until both screws 96 and 106 are locked into position as shown in FIG. 4B. In some embodiments, the flats (shown as 98a-98c) of the head 100 may be one or more flats in order to reduce angle of rotation required to lock the locking screw against the rounded screw. This may assist the operator in preventing excessive rotation of the locking screw and damage to the bony structure of the vertebral body. Having the locking screw 96 and round screw 106 in a configuration as shown in FIG. 4B may enable the screws to be placed closer together enabling use of a smaller width profile stabilizing plate. Such a configuration may also eliminate the need for other types of locking mechanisms.

Steps 66 through 76 may be repeated until the stabilizing plate has been secured to all affected vertebral bodies and all screws have been locked into place. Step 78) Check plate surface and alignment may be then performed by the surgeon comprising a visual and/or tactile inspection and a fluoro check well known to those skilled in the art of orthopedic and/or neurosurgery.

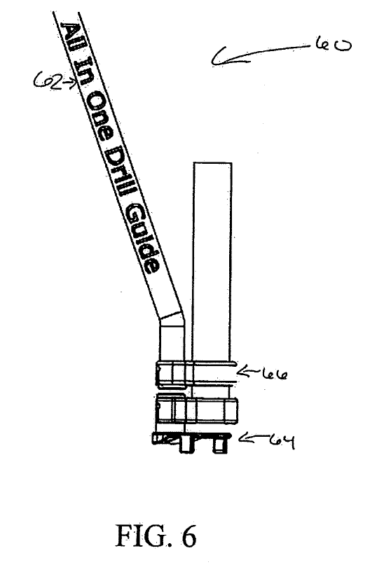

Referring to FIG. 6, an alternate embodiment of the placement and guidance apparatus is shown. The apparatus 60 may include a handle 62 that may be rotatably coupled with the base 64 via a second collar 66 such that the handle 62 may rotate at least 180 degrees for enabling manipulation of the placement and guidance apparatus 60 by a user on either side of the patient and thus the surgical field.

The forgoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

For instance, in some embodiments, there may be a kit 200 provided for performing a surgical procedure to position and implant a stabilizing plate into cervical vertebral bodies within a surgical field. Referring to FIGS. 4, 4A, and 4B, the kit 200 may comprise the following: a placement and guidance apparatus 10 for placing and securing a stabilizing plate into the vertebral bodies comprising an implant engaging base having implant engaging limbs on opposing sides thereof, a handle rotatably secured to the base, an aligning member pivotally coupled about the handle, the aligning member receiving a cannula therein for guiding instruments into the surgical field, wherein the aligning member is pivotally coupled about the handle by a spring-loaded collar enabling the aligning member to be locked into at least two fixed positions; the stabilizing plate comprising multiple pairs of orifices, each orifice for receiving a fastener therethrough; a fixed angle drill guide 82 having a cannula 83 thereon for guiding instruments, tools, and fasteners into the surgical field; a variable angle drill (not shown) guide having a cannula thereon for guiding instruments, tools, and fasteners into the surgical field; a plate holder (not shown); temporary plate holding pins (not shown); a plate bender 80 for bending the stabilizing plate to accommodate a patient's anatomy; an awl 84 for initializing a hole in each vertebral body; at least one drill bit 90a-c for drilling into the hole initialized by the awl 84; at least one tap 86 for further preparing the drilled hole for a fastener; locking screws 96 having flats 98a-c on heads 100 thereof and round screws 106 for placement adjacent to the locking screws, the locking screws 98 frictionally engaging the round screws 100 when the flats 98a-c are turned away from the round screws 100; and a screwdriver 92. Also, the kit 200 may include an implant such as the stabilizing plate 40, as shown in FIG. 2.

In other embodiments, there may be a kit provided for performing a surgical procedure to position and implant a stabilizing plate into cervical vertebral bodies within a surgical field wherein both the locking screws and round screws may be self-drilling.

In yet other embodiments, there may be a kit, provided for performing a surgical procedure to position and implant a stabilizing plate into cervical vertebral bodies within a surgical field wherein both the locking screws and round screws may be self-tapping.

In still other embodiments, there may be a kit provided for performing a surgical procedure to position and implant a stabilizing plate into cervical vertebral bodies within a surgical field wherein the screwdriver may comprise a head having flats thereon shaped identical to the head of the locking screws.

In other embodiments, there may be a kit, provided for performing a surgical procedure to position and implant a stabilizing plate into cervical vertebral bodies within a surgical field comprising a placement and guidance apparatus for placing and securing a stabilizing plate into the vertebral bodies comprising an implant engaging base having implant engaging limbs on opposing sides thereof, a handle rotatably secured to the base, an aligning member pivotally coupled about the handle, the aligning member receiving a cannula therein for guiding instruments into the surgical field, wherein the aligning member is pivotally coupled about the handle by a spring-loaded collar enabling the aligning member to be locked into at least two fixed positions; the stabilizing plate comprising multiple pairs of orifices, each orifice for receiving a screw therethrough; a fixed angle drill guide having a cannula thereon for guiding instruments, tools, and fasteners into the surgical field; a variable angle drill guide having a cannula thereon for guiding instruments, tools, and fasteners into the surgical field; a plate bender for bending the stabilizing plate to accommodate a patient's anatomy; a plate holder; temporary holding pins; an awl for initializing a hole in each vertebral body; at least one drill bit for drilling into the hole initialized by the awl; at least one tap for further preparing the drilled hole for a fastener; a plurality of screws; and a screwdriver.

In other embodiments, there may be a kit provided for performing a surgical procedure to position and implant a stabilizing plate into cervical vertebral bodies within a surgical field wherein the plurality of screws may be self-drilling.

In yet other embodiments, there may be a kit provided for performing a surgical procedure to position and implant a stabilizing plate into cervical vertebral bodies within a surgical field wherein both the plurality of screws may be self-tapping.

What is claimed is:

1. An apparatus for placing and securing a stabilizing plate having multiple pairs of orifices for receiving fasteners to at least two vertebral bodies of a spine, the apparatus comprising:
a base portion rotatably coupled to a handle portion, the handle portion extending away from a first side of the base portion, wherein the base portion comprises a plurality of implant engaging members extending away from a second side of the base portion opposite from the first side, wherein the implant engaging members are configured for grasping the stabilizing plate; and
a pivoting member pivotably coupled to the handle portion above the base portion, wherein the pivoting member comprises a collar having an aligning member such that the collar may be pivoted about the handle to position the aligning member over the implant.

2. The apparatus of claim 1, wherein the implant engaging members comprise limbs which are configured to attach to indentations on the stabilizing plate.

3. The apparatus of claim 1, wherein the collar may be spring-loaded to facilitate locking the aligning member into one or more releasable locked positions over a selected orifice of the implant.

4. The apparatus of claim 1, wherein the handle may rotate at least 180 degrees for enabling manipulation of the apparatus.

5. The apparatus of claim 1, the apparatus further comprising a cannula coupled to the aligning member.

6. A method for securing a stabilizing plate to at least two vertebral bodies, the method comprising:
inserting a locking screw into a first orifice in the stabilizing plate and into a first adjacent orifice in the at least two vertebral bodies, wherein the locking screw has a plurality of flats on a screw head of the locking screw;
turning the locking screw using a screwdriver having a plurality of flats on a handle of the screwdriver which determines the direction of the flats of the locking screw, wherein the locking screw is turned such that one of the three flats of the locking screw is turned to face toward the second adjacent orifice;
inserting a round screw into a second orifice in the plate and into the second adjacent orifice in the at least two vertebral bodies, wherein the round screw has a rounded screw head; and
turning the screw head of the locking screw such that the screw head of the locking screw frictionally engages the rounded head of the round screw.

7. The method of claim 6, further comprising the steps of:
positioning the stabilizing plate above the at least two vertebral bodies;
temporarily coupling a stabilizing plate to the at least two vertebral bodies;
drilling the first adjacent orifice and the second adjacent orifice into the at least two vertebral bodies;

8. The method of claim 6, wherein a plate holder means is used to position the stabilizing plate above the at least two vertebral bodies.

9. The method of claim 8, wherein the step of temporarily coupling the stabilizing plate to the at least two vertebral bodies comprises coupling the stabilizing plate to the at least two vertebral bodies with temporary holding pins.

10. The method of claim 7, further comprising:
coupling the stabilizing plate to a placement and guidance apparatus, wherein the placement and guidance apparatus has engaging members which attach to the stabilizing plate;
inserting the stabilizing plate using the placement and guidance apparatus over the two or more vertebral bodies.

11. The method of claim 10, further comprising:
maintaining the stabilizing plate at the at least two vertebral bodies using the placement and guidance apparatus.

12. The method of claim 11, further comprising guiding a drill to one of the at least two vertebral bodies using a cannula coupled to a pivotable member on the placement and guidance apparatus.

13. A kit for inserting and securing a stabilizing plate into at least two vertebral bodies of the spine, the kit comprising:
a placement and guidance apparatus for placing and stabilizing the stabilizing plate into the vertebral bodies;
a drill guide having a drill guide cannula for guiding instruments, tools, and fasteners;
a stabilizing plate holder;
a plurality of temporary plate holding pins;
a stabilizing plate bender for bending the stabilizing plate;
an awl for initializing a hole in each of the at least two vertebral bodies;
at least one tap for further preparing the hole in each of the at least two vertebral bodies;
a plurality of locking screws having a plurality of flat portions on each of the plurality of locking screws;
a plurality of round screws each having a rounded screw head; wherein each of the plurality of the locking screws are configured frictionally engage the round screws when the flat portions of the locking screws are turned away from the round screws; and
a screwdriver, wherein the screwdriver comprises a shaft having flats dispositioned to align in a coaxial manner to the flats on the heads of the locking screws.

14. The kit of claim 13, the plurality of flat portions comprising two or more flat portions.

15. The kit of claim 14, wherein both the plurality of locking screws and the plurality of round screws are self-drilling.

16. The kit of claim 15, wherein the plurality of locking screws and the plurality of round screws are self-tapping.
